(19) United States
(12) Patent Application Publication
(10) Pub. No.: US 2025/0249022 A1
(43) Pub. Date: Aug. 7, 2025

(54) PHARMACEUTICAL COMPOSITION FOR PREVENTING OR TREATING COLORECTAL CANCER, COMPRISING ESTROGEN AND ANTI-PD-L1 ANTIBODY

(71) Applicants: SEOUL NATIONAL UNIVERSITY R&DB FOUNDATION, Seoul (KR); SEOUL NATIONAL UNIVERSITY HOSPITAL, Seoul (KR)

(72) Inventors: Nayoung KIM, Seoul (KR); Ryoung Hee NAM, Yongin-si (KR); Chin-Hee SONG, Yongin-si (KR)

(73) Assignees: SEOUL NATIONAL UNIVERSITY R&DB FOUNDATION, Seoul (KR); SEOUL NATIONAL UNIVERSITY HOSPITAL, Seoul (KR)

(21) Appl. No.: 18/856,078

(22) PCT Filed: Dec. 20, 2022
(86) PCT No.: PCT/KR2022/020838

§ 371 Date: Oct. 11, 2024

(30) Foreign Application Priority Data
Apr. 12, 2022 (KR) 10-2022-0045193
Jun. 3, 2022 (KR) 10-2022-0068479

(51) Int. Cl. A61K 31/566; A61K 39/00; A61P 35/00; C07K 16/28

(52) U.S. Cl. CPC A61K 31/566; A61P 35/00; C07K 16/2827; A61K 2039/505

(57) ABSTRACT

The present invention relates to a pharmaceutical composition for preventing or treating colorectal cancer comprising estrogen and an anti-PD-L1 antibody, and a composition comprising estrogen for promoting anti-cancer effects of an anti-PD-L1 antibody. Particularly, a combination therapy of estrogen and an anti-PD-L1 antibody carried out in a colorectal cancer model exhibits synergistic effects in tumor growth inhibition that are greater than those of when each is used alone, and induces changes in tumor-related cell populations, and thus is effective as a treatment in precision medicine.

### TECHNICAL FIELD

The present invention relates to a pharmaceutical composition for preventing or treating colorectal cancer comprising estrogen and an anti-PD-L1 antibody, and a composition comprising estrogen for promoting anti-cancer effects of an anti-PD-L1 antibody.

Meanwhile, this application was supported by the following national research and development project.

### NATIONAL RESEARCH AND DEVELOPMENT PROJECT THAT SUPPORTED THIS INVENTION

[Assignment Unique Number] 1711130790
[Assignment Number] 2019R1A2C2085149
[Name of the Ministry] Korea Ministry of Science and ICT
[Assignment management (professional) organization name] National Research Foundation of Korea
[Research Project Name] Individual Basic Research (Korea Ministry of Science and ICT) (R&D)

### [Research Assignment Name]

Effects of sex hormones on immune checkpoints and Nrf2 in the development and progression of colorectal cancer

### [Contribution Rate] 1/1

[Name of project carrying out organization] Seoul National University
[Research period] 2019 Sep. 1-2024 Feb. 29

### BACKGROUND

Colorectal cancer (CRC), the third leading cause of cancer-related death in the United States in 2022, exhibits gender differences in incidence and mortality rates worldwide. The protective effects of estrogen have been reported in a variety of diseases, including neurodegenerative diseases, cardiovascular diseases, and esophageal diseases.

Sex hormones are primarily involved in sexual differentiation and reproduction, but also affect the development and function of immune cells. Men and women differ in their immunological responses to foreign and self-antigens, and differ in their innate and adaptive immune responses. In addition, while some immunological factors (i.e., higher numbers of CD4⁺ T cells and CD4/CD8 T cell ratio in females) remain constant from birth to old age, other immunological factors (i.e., proinflammatory responses) change during puberty. In fact, immune cells such as B cells, T cells, macrophages, monocytes, dendritic cells, and natural killer cells express estrogen receptors, and estrogen regulates the production and function of immune cells.

Meanwhile, immunotherapy has recently emerged as a powerful cancer management tool for conventional cancer treatments such as chemotherapy, radiation, and surgery. In particular, immune checkpoint inhibitors (ICI) have been proven to be effective in extending the survival period of cancer patients due to their low toxicity and high treatment effectiveness. The most widely described ICI targets programmed apoptosis receptor-1 (PD-1), programmed apoptosis receptor-1 ligand (PD-L1), and cytotoxic T lymphocyte-associated proteins (CTLA-4). PD1, which is usually upregulated on the surface of activated T cells, binds to PD-L1 expressed in antigen-presenting cells and other immune cells and receives negative co-stimulating signals to limit T cell activation. In cancer cells, overexpression of PD-L1 induces immune evasion and ultimately forms an immunosuppressive tumor microenvironment (TME). ICI, which blocks PD-1/PD-L1 interactions, has shown remarkable results in cancer treatment and is currently used in combination as a first- or second-line therapeutic agent with a single agent or chemotherapy in lung cancer, malignant melanoma, and gastric cancer, but has not been reported in colorectal cancer. ICI can be used effectively in the treatment of PD-L1-positive tumors and cancers with MSI-H (high microsatellite instability) or combined positive score (CPS), which is defined as the total number of five immune cells. However, very few CRC patients meet these scores and meta-analysis reports exist that the efficacy of ICI is more effective in men or is not related to the sex of patients. To date, studies on gender differences and the influence of the immune environment on the efficacy of ICIs in CRC are lacking.

In addition, Korean Patent Laid-Open Publication No. 10-2015-0091058 presents a means and method for determining whether co-treatment of PD-L1 inhibitors is necessary for breast cancer, but does not disclose what was confirmed in colorectal cancer.

Accordingly, the present inventors completed the present invention by administering estrogen and anti-PD-L1 antibodies in combination in an animal model in a colorectal cancer model.

### SUMMARY OF THE INVENTION

### Technical Problem

Thus, the present inventors hypothesized that estrogen may increase the effectiveness of anti-PD-L1 treatment in colorectal cancer tumor models, and confirmed the effect of combination therapy of estrogen and anti-PD-L1 antibodies in colorectal tumor models, as well as the effect of gender and estrogen on colorectal tumor growth, PD-L1 expression, and tumor-associated cell populations.

Therefore, the present invention is directed to providing a pharmaceutical composition for preventing or treating colorectal cancer, containing estrogen and an anti-PD-L1 antibody.

In addition, the present invention is also directed to providing a composition for enhancing anticancer effects of an anti-PD-L1 antibody on colorectal cancer, containing estrogen.

In addition, the present invention is also directed to providing a method for preventing or treating colorectal cancer comprising administering to a subject in need thereof an effective amount of estrogen and an anti-PL-L1 antibody.

The present invention is also directed to providing a use of estrogen and anti-PD-L1 antibody in the manufacture of a medicament for preventing or treating colorectal cancer.

### Technical Solution

The terms used in the present specification is for descriptive purposes only and should not be construed as limiting. Expressions in the singular include plural expressions unless the context clearly indicates otherwise. It is understood that the terms “comprise” or “have”, when used in the present specification, are intended to specify the presence of stated features, integers, steps, operations, elements, components or a combination thereof described in the specification but not preclude the possibility of the presence or addition of one or more other features, integers, steps, operations, elements, components, or a combination thereof. Unless otherwise defined, all terms used herein, including technical or scientific terms, have the same meaning as commonly understood by one of ordinary skill in the art. Terms such as those defined in a commonly used dictionary should be interpreted as having a meaning consistent with the meaning in the context of the related art, and should not be interpreted in an ideal or excessively formal meaning unless explicitly defined in the present application.

In order to achieve the above objects, the present invention provides a pharmaceutical composition for preventing or treating colorectal cancer, containing estrogen and an anti-PD-L1 antibody.

In one embodiment, the estrogen may be any one or more selected from the group consisting of estradiol, estriol, and estrone.

In one embodiment, the anti-PD-L1 antibody may be any one or more selected from the group consisting of durvalumab, atezolizumab, avelumab, and MDX-1105.

In one embodiment, the pharmaceutical composition may be administered by injection, such as subcutaneous injection, intraperitoneal injection, intravenous injection, or intramuscular injection.

In one embodiment, the weight ratio of the estrogen and the anti-PD-L1 antibody may be 0.5 to 1.5:0.5 to 1.5.

In one embodiment, the pharmaceutical composition may be administered to a male colorectal cancer patient.

In one embodiment, the estrogen may be administered simultaneously, separately, or sequentially with the anti-PD-L1 antibody.

In one embodiment, the estrogen may be administered prior to the anti-PD-L1 antibody.

In one embodiment, the colorectal cancer may be any one or more selected from the group consisting of colon cancer, rectal cancer, colorectal cancer, colon adenocarcinoma, and neuroendocrine tumors.

In one embodiment, the estrogen may be administered in combination with an anti-PD-L1 antibody to exhibit one or more activities selected from the group consisting of a) to b):
a) reduction of tumor-associated macrophages (TAM); and
b) increase in M1 TAM.

In addition, the present invention provides a composition for enhancing anticancer effects of an anti-PD-L1 antibody on colorectal cancer, containing estrogen.

In addition, the present invention also provides a method for preventing or treating colorectal cancer comprising administering to a subject in need thereof an effective amount of estrogen and an anti-PL-L1 antibody.

The present invention also provides a use of estrogen and anti-PD-L1 antibody in the manufacture of a medicament for preventing or treating colorectal cancer.

### Advantageous Effects

When using the pharmaceutical composition for treating colorectal cancer comprising estrogen and anti-PD-L1 according to the present invention, a synergistic effect in tumor growth inhibition is shown compared to when estrogen or anti-PD-L1 is used alone. Estrogen inhibits colorectal cancer tumor growth by decreasing PD-L1 expression and regulating tumor-associated cell populations in male mice. Estrogen may also be used in combination with immune checkpoint inhibitors in combination therapy, as it enhances the antitumor effects of anti-PD-L1 against colorectal cancer tumor growth. The results of the present invention are useful for useful therapeutic strategies for precision medicine, such as co-administration of immune checkpoint inhibitors and estrogen for the treatment of patients with colorectal cancer.

### BRIEF DESCRIPTION OF THE DRAWINGS

FIGS. 1A and 1B show gender differences in MC38 tumor growth. (A and B) are experimental plans, (C) monitored mice for tumor development two or three times per week and measured tumor size using digital calipers, and (D) is a result of weighing resected tumors.

FIGS. 2A and 2B are results of confirming the frequency of PD-L1-positive cells, indicating that they were higher in males than in females. (A) Representative IHC images of MC38 colon tumor sections after PD-L1 staining. Magnification, ×200. Red arrows indicate PD-L1-stained cells. (B) The graph shows the percent frequency of PD-L1 stained cells in male and female mice. (C) Gating strategy to determine PD-L1-expressing cells, and single cells were gated using SSC and FSC. PD-L1-expressing cells were identified in single cell populations (CD274⁺). (D) Representative plots of flow cytometry analysis (left panel) and frequency (right panel) of PD-L1-expressing cells (CD274⁺) in male and female MC38 tumors.

FIGS. 3A-3D are results of confirming the frequency of PD-L1-expressing tumor cells, which was higher in male mice than in female mice. (A) Gating strategy to determine tumor cells, TAI and PD-L1-expressing tumor cells and TAIs, and single cells were gated using SSC and FSC. In single cell populations, cells were identified as tumor cells (CD45⁻), TAIs (CD45⁺), PD-L1-expressing tumor cells (CD45⁻CD274⁺), and PD-L1-expressing TAIs (CD45⁺CD274⁺). (D) Representative plots of flow cytometry analysis (left panel) and frequency (middle and right panels) of tumor cells (CD45⁻) and TAIs (CD45⁺) in male and female MC38 tumors. (C) Representative plots of flow cytometry analysis (left panel) and frequency (middle and right panels) of PD-L1-expressing tumor cells (CD45⁻CD274⁺) and PD-L1-expressing TAIs (CD45⁺CD274⁺) in male and female MC38 tumors. (D) Gating strategy to determine TAMs and PD-L1-expressing TAMs, and single cells were gated using SSC and FSC. TAMs were determined as CD11b⁺F4/80⁺cells in a single cell population, and PD-L1-expressing TAMs were determined as CD274⁺ cells within the TAM population. (E) Representative plots of flow cytometry analysis (left panel) and frequency (right panel) of TAMs (CD11b⁺F4/80⁺) in male and female MC38 tumors. (F) Representative plots of flow cytometry analysis (left panel) and frequency (right panel) of PD-L1-expressing TAMs (CD11b⁺F4/80⁺CD274⁺) in male and female MC38 tumors.

FIGS. 4A-4C show that the frequency of CAFs and PD-L1-expressing TECs was higher in male mice than in female mice. (A) Gating strategy to determine TECs, CAFs and PD-L1-expressing TECs and CAFs, and single cells were gated using SSC and FSC. In the single cell population, CD45⁻ cells were gated, then TECs were determined as CD140a⁻CD31⁺ cells and CAFs were determined as CD140a⁻CD31⁻ cells within the CD45⁻ cell population. PD-L1-expressing TECs and CAFs were determined as CD274⁺ cells within the TEC and CAF populations, respectively. (B) Representative plots of flow cytometry analysis (left panel) and frequency (middle and right panels) of TECs (CD45⁻CD140a⁻CD31⁺) and CAFs (CD45⁻CD140a⁻CD31⁻) in male and female MC38 tumors. (C) Representative plots of flow cytometry analysis (left panel) and frequency (right panel) of PD-L1-expressing TECs (CD45⁻CD140a⁻CD31⁺CD274⁺) in male and female MC38 tumors. (D) Representative plots of flow cytometry analysis (left panel) and frequency (right panel) of PD-L1-expressing CAFs (CD45⁻CD140a⁻CD31⁻CD274⁺) in male and female MC38 tumors.

FIGS. 5A-5C show results on the inhibitory effect of E2 on MC38 tumor growth in male mice. (A and B) are the experimental schemes, (C) mice were monitored 2 or 3 times per week for tumor development, tumor size was measured using digital calipers, and (D) is a result of measuring the weight of the resected tumors.

FIGS. 6A-6H show the inhibitory effect of E2 on tumor-associated cell populations and PD-L1 expression in male MC38 tumors. (A) Representative plots of flow cytometry analysis (top panel) and frequency (bottom panel) of PD-L1-expressing cells (CD274⁺), (B) representative plots of flow cytometry analysis (top panel) and frequency (bottom panel) of tumor cells (CD45) and TAIs (CD45⁺), and (C) representative plots of flow cytometry analysis (top panel) and frequency (bottom panel) of PD-L1-expressing tumor cells (CD45⁻CD274⁺) and PD-L1-expressing TAIs (CD45⁺CD274⁺). (D) Representative plots of flow cytometry analysis of TAMs (CD11b⁺F4/80⁺) (top panel) and PD-L1-expressing TAMs (CD11b⁺F4/80⁺CD274⁺) (bottom panel). (E) Frequency of TAMs (CD11b⁺F4/80⁺), (F) frequency of PD-L1-expressing TAMs (CD11b⁺F4/80⁺CD274⁺), (G) representative plots of flow cytometry analysis (top panel) and frequency (bottom panel) of TECs (CD45⁻CD140a⁻CD31⁺) and CAFs (CD45⁻CD140a⁻CD31⁻), (H) representative plots of flow cytometry analysis (top panel) and frequency (bottom panel) of PD-L1-expressing TECs (CD45⁻CD140a⁻CD31⁺CD274⁺), and (I) representative plots of flow cytometry analysis (top panel) and frequency (bottom panel) of PD-L1-expressing CAFs (CD45⁻CD140a⁻CD31 CD274⁺).

FIGS. 7A-7D show the synergistic effect of E2 on the antitumor effect of anti-PD-L1 antibody on MC38 tumor growth in male mice. (A and B) are the experimental schemes, (C) representative images of MC38 tumor-bearing mice and tumors on day 26, (D) mice were monitored 2 or 3 times per week for tumor development, tumor size was measured using digital calipers, and (E) is a result of measuring the weight of the resected tumors.

FIGS. 8A-8C show that PD-L1 expression was significantly lower in MC38 tumor tissues from the male group treated with E2 or E2+anti-PD-L1 combination. (A) Representative IHC images of MC38 colon tumor sections after PD-L1 staining. Magnification is ×200, and red arrows indicate PD-L1-stained cells. (B) The graph shows the percent frequency of PD-L1 stained cells in male and female mice, and (C) representative plots of flow cytometry analysis (left panel) and frequency (right panel) of PD-L1-expressing cells (CD274⁺) in male and female MC38 tumors.

FIGS. 9A-9G show the synergistic effect of E2 on alterations of TAM and M1 TAM populations in male mice by anti-PD-L1 antibodies. (A) Representative plots of flow cytometry analysis (left panel) and frequency (right panel) of CD45⁻ tumor cells in male and female MC38 tumors, (B) representative plots of flow cytometry analysis (left panel) and frequency (right panel) of PD-L1-expressing tumor cells (CD45⁻CD274⁺) in male and female MC38 tumors, and (C) Gating strategy to determine TAMs and M1 TAMs, PD-L1-expressing TAMs and M1 TAMs, and single cells were gated using SSC and FSC. TAMs were determined as CD11b⁺F4/80⁺ cells in a single cell population, and M1 TAMs were determined as CD86⁺ cells within the TAM population. PD-L1-expressing TAMs and M1 TAMs were determined as CD274⁺ cells within the TAM and M1 TAM populations, respectively. (D) Representative plots of flow cytometry analysis (left panel) and frequency (right panel) of TAMs (CD11b⁺F4/80⁺) in male and female MC38 tumors. (E) Representative plots of flow cytometry analysis (left panel) and frequency (right panel) of M1 TAMs (CD11b⁺F4/80⁺CD86⁺) in male and female MC38 tumors. (F) Representative plots of flow cytometry analysis (left panel) and frequency (right panel) of PD-L1-expressing TAMs (CD11b⁺F4/80⁺CD274⁺) in male and female MC38 tumors. (G) Representative plots of flow cytometry analysis (left panel) and frequency (right panel) of PD-L1-expressing M1 TAMs (CD11b⁺F4/80⁺CD86⁺CD274⁺) in male and female MC38 tumors.

FIG. 10 shows the proposed estrogen regulatory mechanism on MC38 tumors in male mice. (A) shows the regulatory factors of PD-L1 expression in the tumor microenvironment. PD-L1 abundance is regulated by several transcription factors, including HIF1α, MYC, STAT3, AP1, NF-κB, and NRF2. PD-L1 expressed on tumor cells binds to PD-1 on T cells and is involved in immune evasion. (B) shows the mechanism of inhibition of PD-L1 abundance by co-treatment with E2 and anti-PD-L1 antibodies. ERB activated by E2 binding translocates to the nucleus and inhibits PD-L1 transcriptional activity by inhibiting NF-κB activated by the TLR signaling pathway and NRF2 activated by the GPCR signaling pathway. Anti-PD-L1 antibodies bind to PD-L1 and inhibit PD-L1 binding to PD-1 on T cells.

### DETAILED DESCRIPTION OF THE EMBODIMENTS

Hereinafter, the present invention will be described in more detail.

As described above, the inventors hypothesized that estrogen may increase the effectiveness of anti-PD-L1 therapy in colorectal cancer models, and evaluated the effects of combination therapy of estrogen and anti-PD-L1 antibody in colorectal cancer models and the expression levels of relevant cell populations, PD-L1 expression levels, and cancer-associated fibroblasts and tumor cells in colorectal cancer models. In addition, the present invention has been completed by confirming the level of macrophages and confirming that a synergistic effect was generated compared to when treated alone.

Therefore, the present invention provides a pharmaceutical composition for preventing or treating colorectal cancer, comprising estrogen and an anti-PD-L1 antibody.

In the present invention, the term “pharmaceutical composition” means a mixture comprising an anti-PD-L1 antibody comprising estrogen of the present invention and a pharmaceutically acceptable excipient such as a diluent or carrier. In some embodiments, a method is provided for administering to a subject in need thereof a pharmaceutical composition comprising an anti-PD-L1 antibody comprising estrogen of the present invention. In some embodiments, the compositions of the present invention can be administered to humans. Although the description of pharmaceutical compositions provided herein is principally directed to pharmaceutical compositions for administration to humans, those skilled in the art will appreciate that such compositions are generally suitable for administration to all types of animals. A skilled veterinary pharmacologist who understands the modifications of pharmaceutical compositions for administration to various animals can design and/or perform such modifications, if necessary, simply by routine experimentation.

A pharmaceutically acceptable salt refers to a composition that is physiologically acceptable and does not typically cause allergic reactions such as gastrointestinal upset, dizziness, or similar reactions when administered to humans. Examples of the carriers, excipients and diluents include lactose, dextrose, sucrose, sorbitol, mannitol, xylitol, erythritol, maltitol, starch, acacia gum, alginate, gelatin, calcium phosphate, calcium silicate, cellulose, methyl cellulose, polyvinylpyrrolidone, water, methyl hydroxybenzoate, propyl hydroxybenzoate, talc, magnesium stearate and mineral oil. In addition, it may further include fillers, anticoagulants, lubricants, wetting agents, fragrances, emulsifying agents, and preservatives.

The compositions of the present invention can be formulated using methods known in the art so as to provide rapid, sustained or delayed release of the active ingredient after administration to a mammal. The formulation may be in the form of powder, granule, tablet, emulsion, syrup, aerosol, soft or hard gelatin capsule, sterile injectable solution, or sterile powder.

“Treatment” means an approach aimed at obtaining beneficial or desirable clinical outcomes. For the purposes of the present invention, beneficial or desirable clinical outcomes include, but are not limited to, palliation of symptoms, reduction of disease scope, stabilization of disease condition (i.e., no exacerbation), delay or reduction of disease progression, improvement or temporary palliation and mitigation of disease condition (partially or entirely), and whether detectable or undetectable. In addition, “treatment” may also mean increasing survival rate compared to the expected survival rate when not treated. “Treatment” refers to both therapeutic treatment and preventive or prophylactic measures. The above treatments include not only the treatments required for the disorders to be prevented, but also the treatments required for the disorders that have already occurred. “Palliating” a disease means reducing the extent of the disease state and/or its unfavorable clinical manifestations and/or slowing or lengthening the time course of its progression, compared to the absence of treatment.

In some embodiments, the pharmaceutical composition for preventing or treating colorectal cancer containing estrogen and an anti-PD-L1 antibody of the present invention may be administered in combination with any therapeutic active agent or procedure (e.g., surgery, radiation therapy) that is useful for treating, mitigating, improving, and palliating one or more symptoms or cancer features, delaying initiation, inhibiting its progression, reducing its severity, and/or reducing its incidence.

In one embodiment, the estrogen may be any one or more selected from the group consisting of estradiol, estriol, and estrone, and the anti-PD-L1 antibody may be any one or more selected from the group consisting of durvalumab, atezolizumab, avelumab, and MDX-1105, but is not limited thereto.

Specifically, estrogen was considered a steroid hormone that was first discovered as a substance that regulates the development and growth of human reproductive organs, but later studies have shown that estrogen receptors are involved in other physiological and pathological processes in the cardiovascular, skeletal, and neuroendocrine systems other than the reproductive system. That is, estrogen exhibits genetic or non-genomic effects through estrogen receptors, and these estrogen receptors are largely divided into ERa and ERb. ERa is mainly distributed in female reproductive organs such as the breasts, uterus, and ovaries, whereas ERb is more widely distributed in various organs of our body and has been found to have different functions from ERa. Estrogen can have a variety of effects on various organs, such as increasing the risk of breast cancer and endometrial cancer, or decreasing the risk of colorectal cancer. In the present invention, the estrogen may preferably be estradiol, and more preferably 17b-estradiol among estradiol.

The anti-PD-L1 antibody refers to a substance that attacks cancer cells by activating T cells by blocking the activation of immune checkpoint proteins involved in T cell suppression. The anti-PD-L1 antibody may bind to a protein expressed on the surface of cancer cells and may specifically act as a PD-L1 inhibitor.

As used in the present invention, “antibody” is a term known in the art and refers to a specific protein molecule directed against an antigenic site. For the purposes of the present invention, an antibody means an antibody that specifically binds to the PD-L1 protein of the present invention, and such an antibody can be produced by conventional methods by cloning each gene into an expression vector according to conventional methods to obtain a protein encoded by said PD-L1 gene, and by conventional methods from the obtained protein. This also includes partial peptides that can be made from said proteins, wherein the partial peptides of the present invention includes at least 7 amino acids, preferably 9 amino acids, more preferably 12 or more amino acids. The form of the antibodies of the present invention is not particularly limited, and polyclonal antibodies, monoclonal antibodies or parts thereof, provided they have antigen binding properties, are also included in the antibodies of the present invention, and all immunoglobulin antibodies are included. Furthermore, the antibodies of the present invention include special antibodies, such as humanized antibodies. The antibody to the protein encoded by the PD-L1 gene of the present invention may be any antibody that can be produced by methods known in the art. For example, an anti-PD-L1 antibody of the present invention may include a functional fragment of an antibody molecule as well as a complete form having two full-length light chains and two full-length heavy chains. The functional fragment of an antibody molecule means a fragment that possesses at least antigen-binding functionality and may be, but is not particularly limited to, Fab, F(ab′), F(ab′)2, Fv, and the like.

In addition, “immuno-oncology” refers to a therapeutic agent that help boost a patient's immune system to fight cancer instead of treatments or drugs that directly attack cancer cells, such as radiation or chemotherapy. It works primarily by looking for immune checkpoint proteins (PD-1, PDL1, CTLA-4) to help T-cells (immune cells) destroy cancer cells by paralyzing their ability to evade immunity. It works to activate the body's immune system to attack cancer cells, so it can be applied to a variety of cancers, and it can also reduce side effects such as indigestion, vomiting, leukopenia, and hair loss. A variety of antibodies that block immune checkpoint proteins are currently in clinical use or in clinical trials. The present invention utilizes anti-PD-L1 as an immuno-oncology.

The pharmaceutical compositions of the present invention may be administered by any route. In some embodiments, the pharmaceutical composition is administered by various routes including intravenous, intramuscular, intraarterial, intramedullary, intraspinal, subcutaneous, intraventricular, percutaneous, intradermal, rectal, intravaginal, intraperitoneal, topical (by powder, ointment, cream, and/or droplets), mucosal, sublingual; intratracheal drip injection, bronchial drip injection, and/or inhalation. Routes specifically considered include penetrating intravenous injection, local administration through blood and/or lymphatic supply, and/or direct administration to the affected area. In general, the most appropriate route of administration will depend on a variety of factors, including the properties of the agent (e.g., stability in the environment of the gastrointestinal tract) and the disorder of the subject. However, the present invention encompasses delivery of the pharmaceutical composition according to the present invention by any suitable route, taking into account advances in the field of drug delivery.

In one embodiment, the pharmaceutical composition may be administered by injection, such as subcutaneous injection, intraperitoneal injection, intravenous injection, or intramuscular injection, but may be administered without limitation by any method that is most effective and has no side effects.

In certain embodiments, the pharmaceutical composition comprising estrogen and an anti-PD-L1 antibody of the present invention for preventing or treating colon cancer may be administered at a dosage level sufficient to deliver from about 0.001 mg/kg to about 100 mg/kg, from about 0.01 mg/kg to about 50 mg/kg, from about 0.1 mg/kg to about 40 mg/kg, from about 0.5 mg/kg to about 30 mg/kg, from about 0.01 mg/kg to about 10 mg/kg, from about 0.1 mg/kg to about 10 mg/kg, or from about 1 mg/kg to about 25 mg/kg of body weight of the subject once or more per day to achieve the desired therapeutic effect. The target dosage may be delivered three times daily, twice daily, once daily, every other day, every three days, every week, every two weeks, every three weeks, or every four weeks. In certain embodiments, the target dosage may be delivered via multiple administrations (e.g., 2, 3, 4, 5, 6, 7, 8, 9, 10, 11, 12, 13, 14 or more administrations).

In one embodiment, the weight ratio of the estrogen and the anti-PD-L1 antibody may be 0.5 to 1.5:0.5 to 1.5, preferably 1:1, and may be appropriately adjusted by a person skilled in the art, but is not limited thereto. In a specific embodiment, as shown in Example 6, estrogen and anti-PD-L1 were each administered at a dose of 10 mg/kg.

In one embodiment, the pharmaceutical composition may be administered to a male colorectal cancer patient.

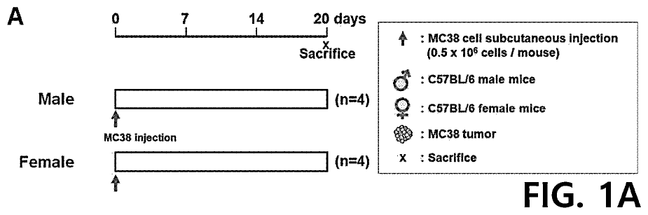

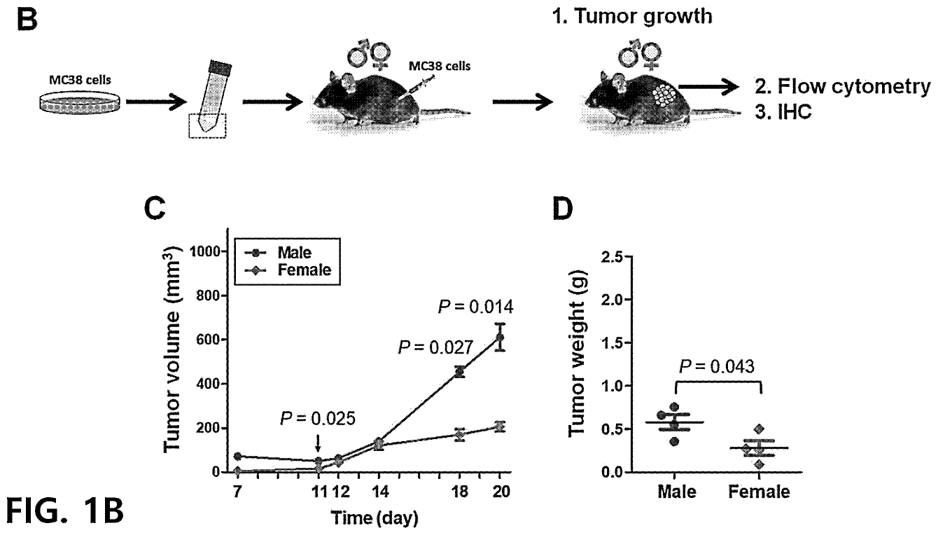

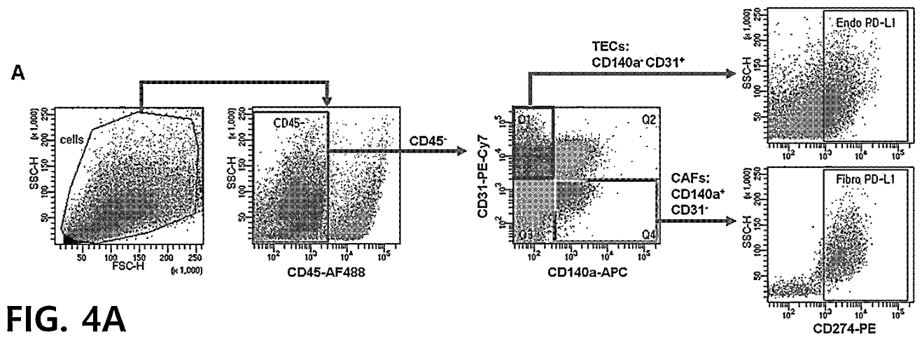

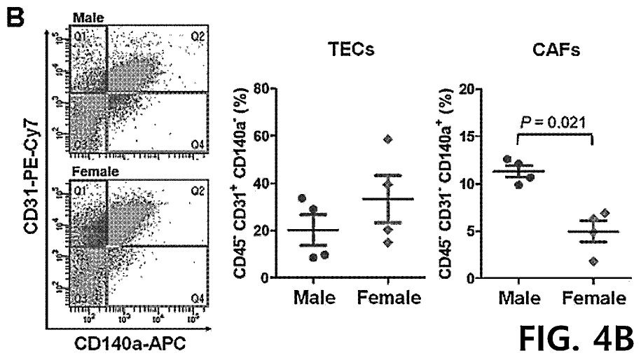

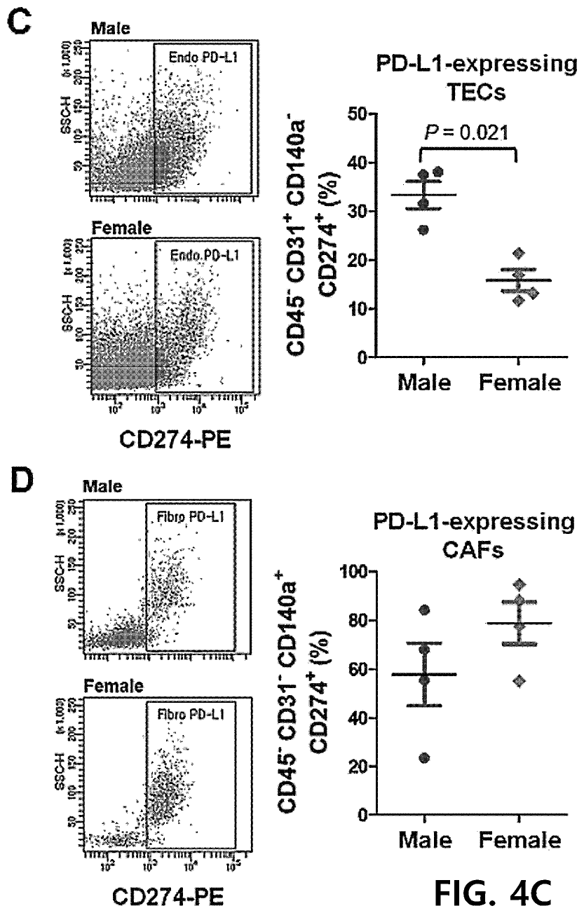

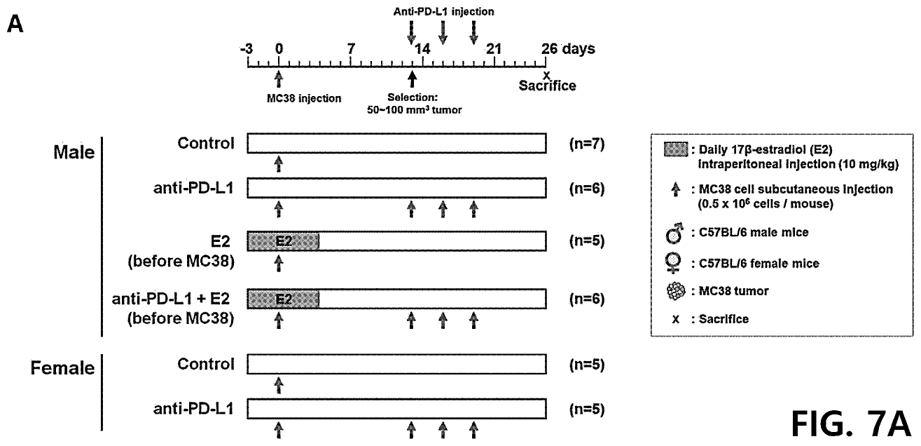

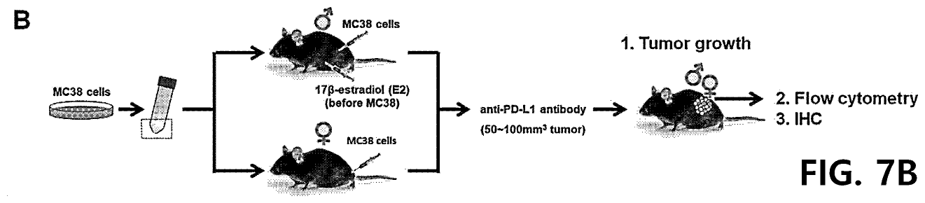

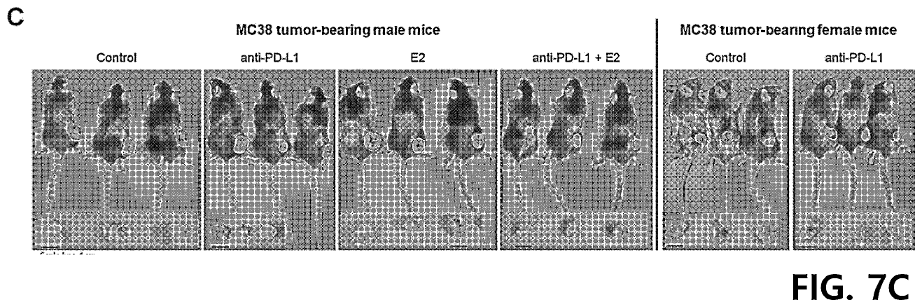

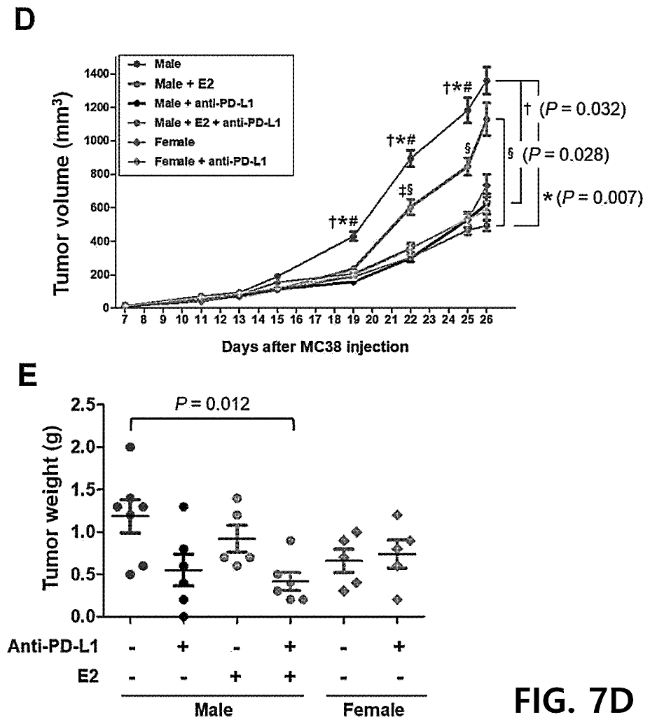

In a specific embodiment, we confirmed that gender was an important factor in colorectal cancer growth (FIGS. 1A-1B), and a higher proportion of TECs expressing PD-L1 contributed to tumor growth in male mice (FIG. 4C). In addition, tumor growth was significantly reduced after anti-PD-L1 antibody treatment in male mice, and tumor growth was significantly slower and smaller with estrogen and anti-PD-L1 treatment (FIGS. 7A to 7D). That is, it was confirmed that when male mice were treated with a pharmaceutical composition containing estrogen and anti-PD-L1 antibodies, tumor growth was significantly inhibited.

In one embodiment, the estrogen may be administered simultaneously, separately, or sequentially with the anti-PD-L1 antibody. Alternatively, in one embodiment, the estrogen may be administered prior to the anti-PD-L1 antibody.

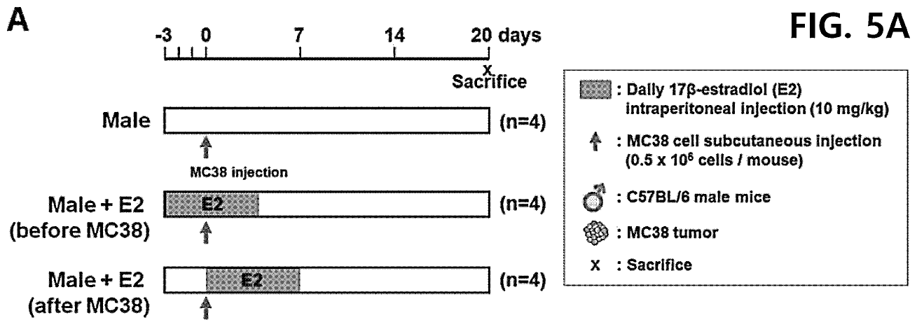

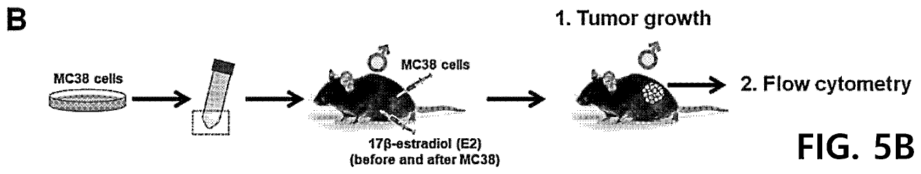

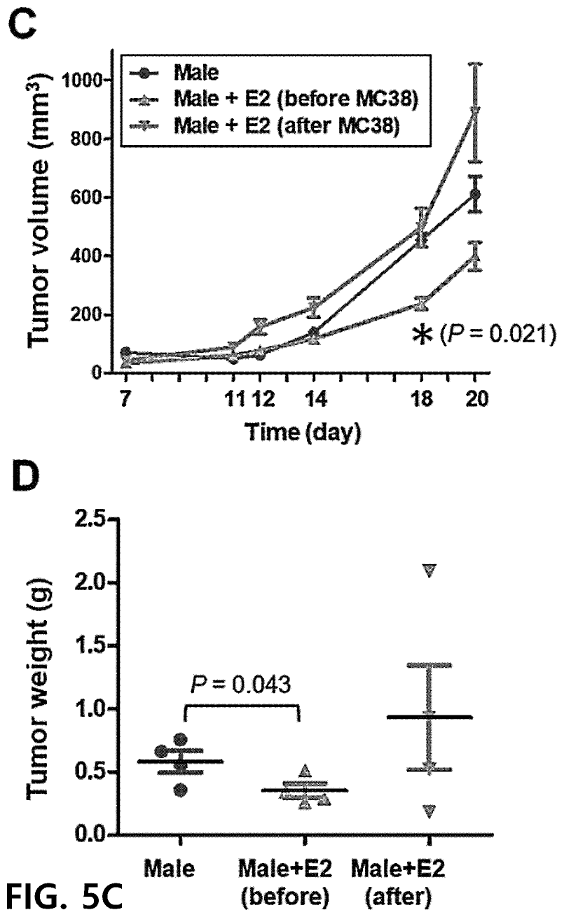

In a specific embodiment, the effect of estrogen treatment time was confirmed, and it was confirmed that estrogen administered first was important for tumor growth inhibition, as shown in FIG. 5A. It was confirmed that the tumor size was significantly reduced when estrogen treatment was performed before administering anti-PD-L1 antibodies (FIGS. 7A-7D).

In one embodiment, the colorectal cancer may be any one or more selected from the group consisting of colon cancer, rectal cancer, colorectal cancer, colon adenocarcinoma, and neuroendocrine tumors, but is not limited thereto.

Meanwhile, tumor associated macrophages (TAMs) are known to be macrophages involved in the growth, proliferation, and metastasis of cancer cells in the tumor (cancer) microenvironment. Macrophages are concentrated around the tumor and penetrate into it, and if a large number of TAMs exist around the tumor in cancer patients, the prognosis and survival rate of patients are reported to be poor. Since TAM contributes to angiogenesis and enables tumor deterioration by inhibiting anti-cancer immune action, the elimination of TAM in cancer treatment is considered important.

TAMs are differentiated into M1 macrophages and M2 macrophages by tumor (cancer) microenvironment. Macrophages are activated into the M1 type, which induces inflammatory responses, and the M2 type, which suppresses immune responses, and TAMs share the characteristics of the M2 type. M1 macrophages are also called CLS macrophages (Crown-like structure macrophages) and perform the function of causing cancer cell death and reducing tumor proliferation, and M2 macrophages, also called as resident macrophages, are known to induce angiogenesis in the cancer microenvironment and cause cancer cell metastasis, unlike M1 macrophages.

In one embodiment, the estrogen may be administered in combination with an anti-PD-L1 antibody to exhibit one or more activities selected from the group consisting of a) to b):
a) reduction of tumor-associated macrophages (TAM); and
b) increase in M1 TAM.

The term “administered in combination” means that the compounds or ingredients are administered together (co-administered) to the subject animal. The co-administration of each compound or ingredient means that each ingredient can be administered at the same time, in any order, or sequentially at different times, to achieve the desired therapeutic effect.

The term “synergy” refers to the fact that the effect of each ingredient when administered together (in combination) is greater than the sum of the effects of each ingredient when administered alone.

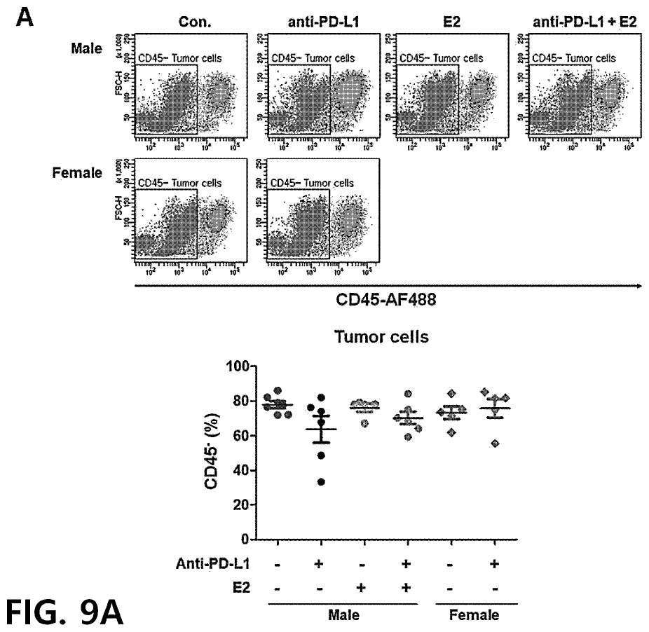

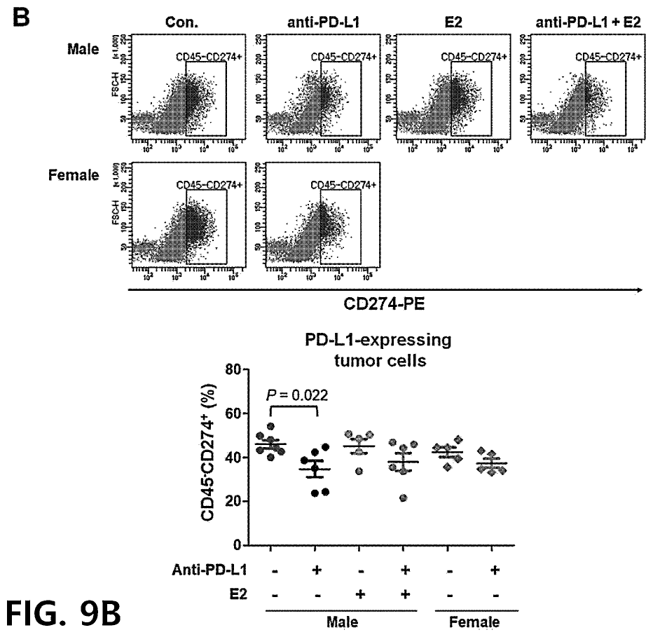

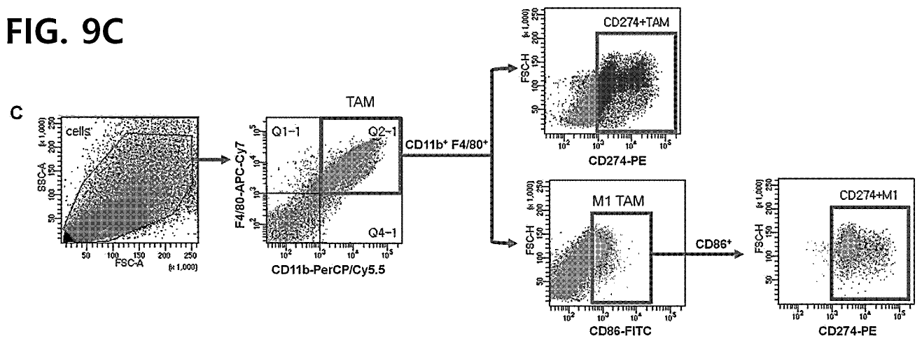

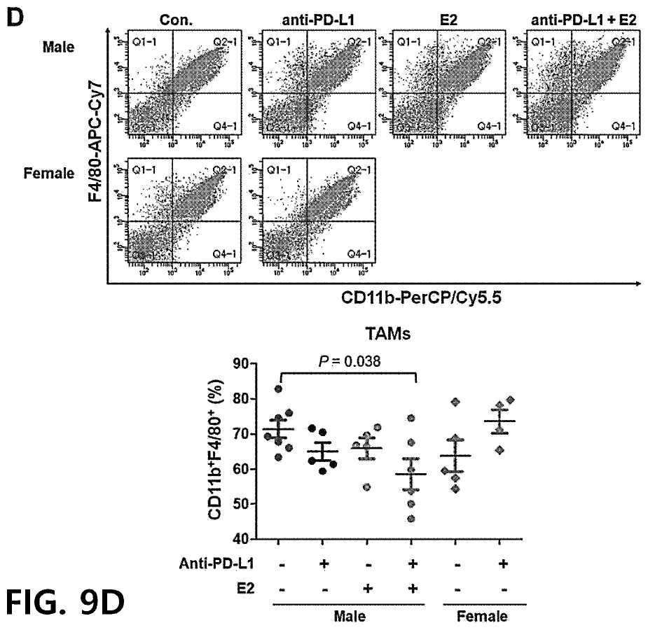

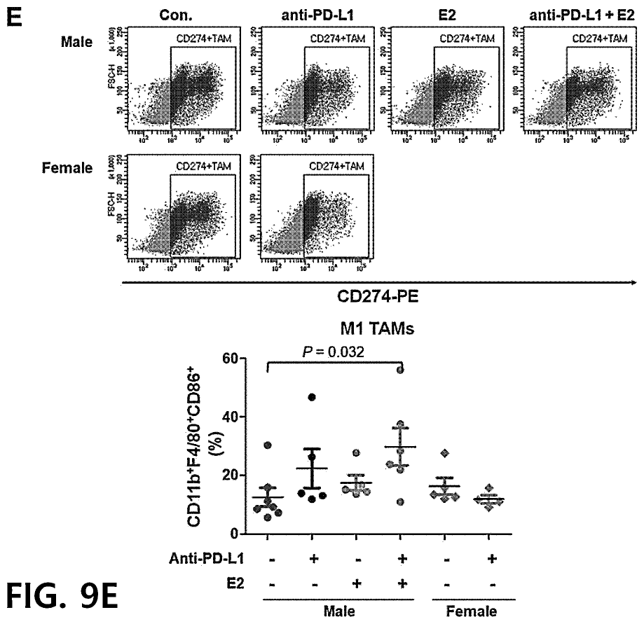

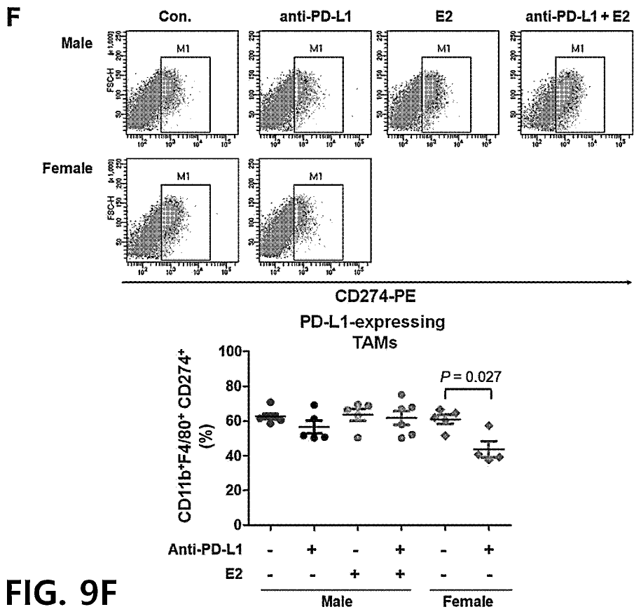

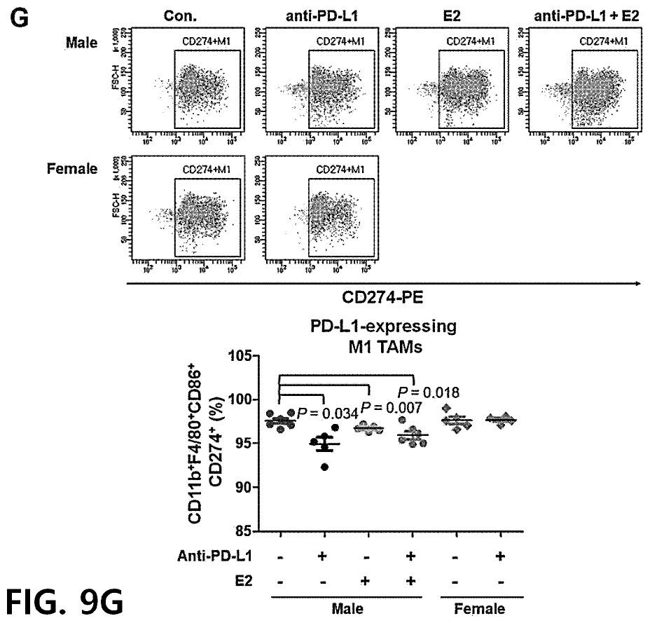

In a specific embodiment, as shown in FIGS. 9D and 9E, when estrogen or anti-PD-L1 antibody was administered in combination, TAM was significantly reduced and M1 TAM was increased, confirming that tumor proliferation was inhibited compared to when estrogen or anti-PD-L1 antibody was administered alone.

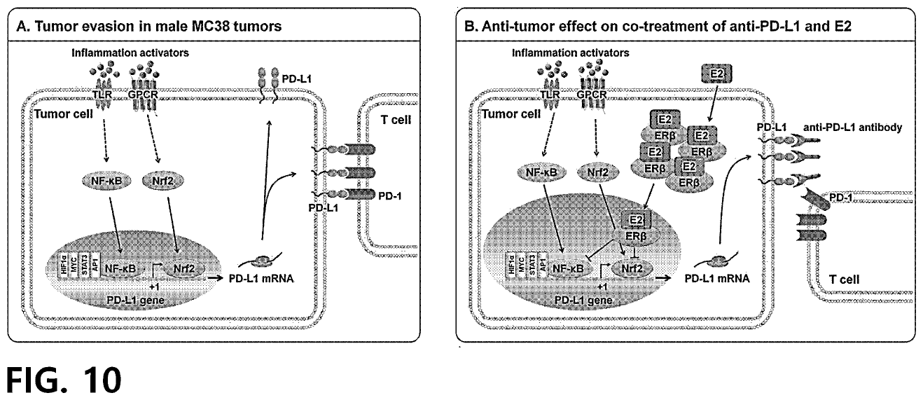

In this regard, FIG. 10 shows the mechanism of inhibition of PD-L1 abundance by co-treatment with estrogen and anti-PD-L1 antibody. ERB activated by estrogen (represented by E2) binding translocates to the nucleus and inhibits PD-L1 transcriptional activity by inhibiting NF-κB activated by the TLR signaling pathway and NRF2 activated by the GPCR signaling pathway. Anti-PD-L1 antibodies exhibit antitumor effects by binding to PD-L1 and inhibiting PD-L1 binding to PD-1 on T cells.

In addition, the present invention provides a composition for enhancing anticancer effects of an anti-PD-L1 antibody on colorectal cancer, containing estrogen.

Regarding the composition for enhancing anticancer effects of colorectal cancer, the above-mentioned description that overlaps with the pharmaceutical composition for preventing or treating colorectal cancer is omitted.

In addition, the present invention also provides a method for preventing or treating colorectal cancer comprising administering to a subject in need thereof an effective amount of estrogen and an anti-PL-L1 antibody.

Regarding the method for preventing or treating colorectal cancer, the above-mentioned description that overlaps with the pharmaceutical composition for preventing or treating colorectal cancer is omitted.

In one embodiment, the subject may be a male colorectal cancer patient.

The present invention also provides a use of estrogen and anti-PD-L1 antibody in the manufacture of a medicament for preventing or treating colorectal cancer.

Regarding the use of estrogen and anti-PD-L1 antibody in the manufacture of a medicament for preventing or treating colorectal cancer, the above-mentioned description that overlaps with the pharmaceutical composition for preventing or treating colorectal cancer is omitted.

Hereinafter, the present invention will be described in more detail through examples. These examples are only for illustrating the present invention, and it will be obvious to those skilled in the art that the scope of the present invention is not to be construed as limited by these examples.

### Preparation Example

### Cell Culture

MC38 cells purchased from Kerafast Inc. (#ENH204-FP, Boston, MA, USA) were maintained in Dulbecco's modified Eagle medium (DMEM) supplemented with 95% humidified atmosphere containing 5% CO2, 10% fetal bovine serum (FBS), 2 mM L-glutamine, 1 mM sodium pyruvate, 10 mM HEPES, and an antibiotic-antifungal mixture at 37° C. All media were purchased from Gibco BRL (Thermo Fisher Scientific, Inc., Waltham, MA, USA). For transplantation, MC38 cells (passage number 7-8) were washed with Dulbecco's phosphate-buffered saline (DPBS), treated slightly with trypsin, washed with DPBS, and checked for viability before final resuspension in the desired concentration of DPBS.

### Animal Model

Male and female C57BL/6 mice aged 7 weeks were obtained from Orient Bio Inc. (Seoul, Korea) and maintained under specific pathogen-free conditions at 23° C. on a light/dark cycle (12/12 h). All mice were randomly divided by sex, weighed, grouped, and housed in filter-top cages, 3-5 mice per cage, in the same room. Animals were marked with an ear punch to allow individual mice to be tracked throughout the experimental period. The preclinical animal room was managed by qualified personnel in accordance with the Animals in Research: Reporting In Vivo Experiments (ARRIVE) guidelines. All animal experiments were approved by the Institutional Animal Care and Use Committee (IACUC) of Seoul National University Bundang Hospital (Approval No. BA-2013-316-023-01). All experiments were performed in accordance with the relevant guidelines and regulations of the Animal Care and Use Committee of Seoul National University Bundang Hospital Animal Hospital and the recommendations of the ARRIVE guidelines.

### Flow Cytometry

Cells were isolated from tumors using the Mouse Tumor Dissociation Kit (#130-096-730, Miltenyi Biotec, Germany) and the Gentle MACS™ Octo Dissociator with Heaters (#130-096-427, Miltenyi Biotec) according to the manufacturer's instructions. All steps were performed on ice. Tumor tissues were cut into 2-4 mm pieces and transferred to GentleMax™ C tubes (#130-096-334, Miltenyi Biotec) containing the enzyme mixture prepared according to the manufacturer's instructions. The tubes containing the samples were then cultivated in a GentleMACS™ Octo Dissociator at 37° C. for 40 minutes (program: 37C_m_TDK_1). After centrifugation, the digested cell suspension was filtered through a 70-μm cell filter (#352350, BD Biosciences, USA). Next, single cell suspensions were incubated with Fc receptor blocking antibody (#156604, BioLegend Inc., San Diego, CA, USA) for 30 minutes to prevent nonspecific antibody binding and then stained with fluorescently labeled specific antibodies. Detailed information on the antibodies used is given in [Table 1].

TABLE 1

| | | | | | | |
|---|---|---|---|---|---|---|
| | | | | Conc. | | Supplier, Cat. |
| Antigen | Label | Clone | Isotype | (ml/ml) | Dilution | no., Lot no. |

In vivo treatment

| | | | | | | |
|---|---|---|---|---|---|---|
| PD-L1 | — | 10F.9G2 | Rat IgG2b, κ | 6.6 | 10 | BioXcell, |
| | | | | | mg/kg | Be0101, |
| | | | | | | 751220D1B |
| Isotype | — | LTF-2 | Rat IgG2b, κ | 9.45 | 10 | BioXcell, |
| control | | | | | mg/kg | Be0090, |
| | | | | | | 767920D1 |

Flow cytometry

| | | | | | | |
|---|---|---|---|---|---|---|
| CD274 (PD- | PE | 10F.9G2 | Rat IgG2b, κ | 0.2 | 1:100 | Biolegend, |
| L1) | | | | | | 124308, B318306 |
| CD45 | AF488 | 30-F11 | Rat IgG2b, κ | 0.5 | 1:200 | Biolegend, |
| | | | | | | 103122, B285085 |
| CD11b | PerCP/Cy5.5 | M1/70 | Rat IgG2b, κ | 0.2 | 1:50 | Biolegend, |
| | | | | | | 101228, B308468 |
| F4/80 | APC-Cy7 | BM8 | Rat IgG2a, κ | 0.2 | 1:50 | Biolegend, |
| | | | | | | 123118, B335934 |
| CD140a | APC | APA5 | Rat IgG2a, κ | 0.2 | 1:25 | Biolegend, |
| | | | | | | 135908, B257183 |
| CD31 | PE-Cy7 | 390 | Rat IgG2a, κ | 0.2 | 1:100 | Biolegend, |
| | | | | | | 102418, B312607 |
| CD86 | FITC | GL1 | Rat IgG2a, κ | 0.5 | 1:50 | BD Biosciences, |
| | | | | | | 553691, 1032076 |

Immunohistochemistry

| | | | | | |
|---|---|---|---|---|---|
| PD-L1 | — | M1H5 | Rat IgG2a, λ | 1:50 | eBioscience, |
| | | | | | 14-5982-82, |
| | | | | | 2110796 |

Data acquisition was performed on a ED FACSCalibur™ or BD FACSAria™ III flow cytometer (BD Biosciences), and data analysis was performed using BD FACSDiva Software (BD Biosciences, version 9.0.1). Cell subtypes were defined by the presence (indicated by ⁺) or absence (indicated by ⁻) of markers expressed on the cell surface. The gating strategies used are listed in [Table 2].

TABLE 2

| Cell population | Cell surface marker |
|---|---|
| Tumor cell | CD45⁻ |
| TAI | CD45⁺ |
| TAM | CD11b⁺ F4/80⁺ |
| TEC | CD45⁻ CD140a⁻ CD31⁺ |
| CAF | CD45⁻ CD140a⁻ CD31⁻ |
| M1 TAM | CD11b⁺ F4/80⁺ CD86⁺ |
| PD-L1-expressing cell | CD274⁺ |
| PD-L1-expressing tumor cell | CD45⁻ CD274⁺ |
| PD-L1-expressing TAI | CD45⁺ CD274⁺ |
| PD-L1-expressing TAM | CD11b⁺ F4/80⁺ CD274⁺ |
| PD-L1-expressing TEC | CD45⁻ CD140a⁻ CD31⁺ CD274⁺ |
| PD-L1-expressing CAF | CD45⁻ CD140a⁻ CD31⁻ CD274⁺ |
| PD-L1-expressing M1 TAM | CD11b⁺ F4/80⁺ CD86⁺ CD274⁺ |

TAI, tumor-associated immune cell; TAM, tumor-associated macrophage; TEC, tumor-associated endothelial cell; CAF, cancer-associated fibroblast; PD-L1, programmed death-ligand 1; CD, cluster of differentiation.

### Statistical Analysis

Statistical analysis was performed using PASW Statistics 18 software (SPSS. Chicago. IL. USA). Data in the scatterplot are presented as mean±standard error of the mean (SEM). Statistical significance of differences between the two groups was assessed using the Mann-Whitney test, and graphs were generated using GraphPad Prism 5.0 (GraphPad Software, San Diego, USA). Statistical significance was set at P<0.05.

### Example 1

Confirmation of Larger MC38 Tumor Growth in Male Mice than in Female Mice

To determine whether the sex of the host affects the growth of transplanted colon tumor cells, a total of 5×10⁵ MC38 cells (passage number 7) resuspended in 100 μl DPBS were injected subcutaneously into the right flank of 8-week-old C57BL/6 male and female mice. Mice were sacrificed 205 days after MC38 cell injection (FIG. 1A), and the collected tumor samples were used for flow cytometry and PD-L1 IHC analysis (FIG. 1B). Specifically, five mice from each group were used for MC38 cancer cell transplantation, and one mouse from each group that did not develop a tumor was excluded from the experiment. Therefore, in the experiment described in FIG. 1A, results were obtained from 8 out of 10 mice in total.

Tumor growth in mice was monitored 2 to 3 times per week, and tumor size was measured using digital calipers. Tumor volume was calculated using the formula (minimum diameter) 2×(maximum diameter)×0.5. The weight of each resected tumor on the day of sacrifice was measured and used for subsequent analyses. The same tumor was divided into two parts to prepare samples for flow cytometry and immunohistochemistry, and experiments were performed.

As a result, male mice showed significantly increased MC38 tumor growth compared to female mice, and the difference increased over time (FIG. 1C). Tumor weights were also significantly higher in male mice than in female mice (FIG. 1D; P=0.043). These results confirmed that the host's sex is an important factor in the growth of colorectal cancer.

### Example 2

Confirmation of Higher Expression of PD-L1 in MC38 Tumors from Male Mice than from Female Mice

Next, whether the expression of PD-L1 and the PD-L1-expressing cell population were affected by gender was investigated. To quantify PD-L1 expression, IHC analysis for PD-L1 staining was performed on MC38 tumor tissues from male and female mice.

### Immunohistochemical (IHC) Analysis of PD-L1 Expression

Tumor tissues were fixed with 4% paraformaldehyde and embedded in paraffin. IHC for PD-L1 was performed using the BenchMark XT automated slide staining system (Ventana Medical Systems, Tucson, AZ, USA) and the ultraView Universal DAB Detection Kit (Ventana Medical Systems) according to the manufacturer's instructions. Each section was blocked with 3% hydrogen peroxide and normal serum. The sections were then incubated with anti-PD-L1 antibody (#14-5982-82, eBioscience, San Diego, USA). Section slides were imaged using a Pannoramic 250 Flash III digital slide scanner (3DHISTECH Ltd., Budapest, Hungary). Low-power 1 field (magnification, 40×) images were acquired automatically. PD-L1-expressing cells were quantitatively evaluated using the Image-Pro Plus analysis system. Within the acquired low-magnification images, three randomly selected high-magnification fields (magnification, 200×) were used to calculate the average number of cells stained for PD-L1 out of the total number of cells. Nonspecific PD-L1 staining was manually excluded. The reported values are the average of different images from different mice within each group. The values on the y-axis are expressed as the percentage of cells stained for PD-L1 out of the total number of cells.

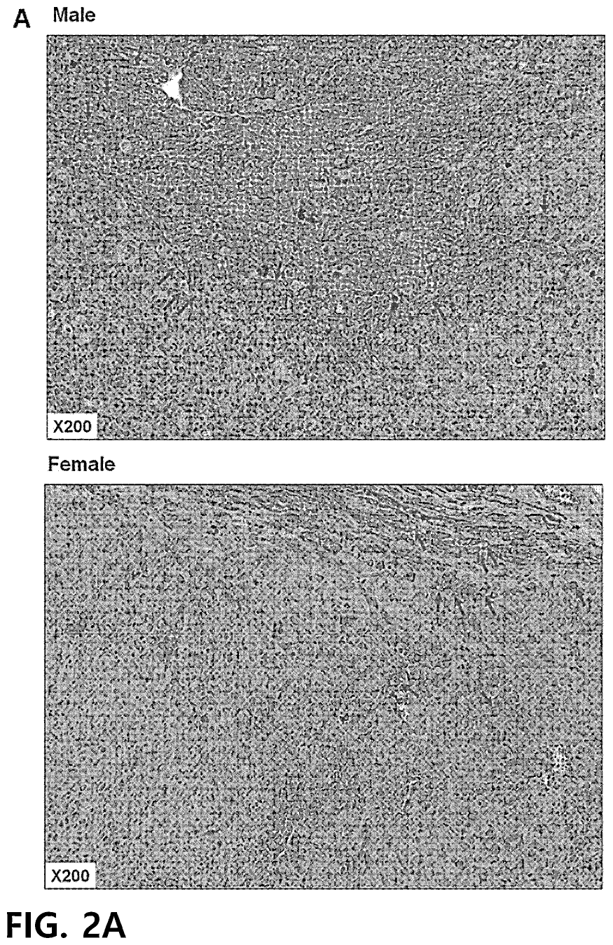

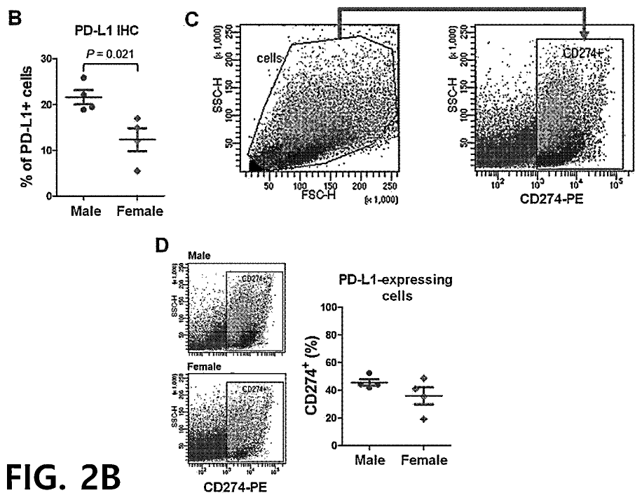

As a result, as shown in FIG. 2A, a representative IHC image, the frequency of PD-L1 positive cells was significantly higher in male mice than in female mice (FIG. 2B; P=0.021).

In addition, flow cytometry was performed to quantify PD-L1 expression. The gating strategy for stained cells is as shown in FIG. 2C. The percentage of PD-L1-expressing cells in the total cell population was higher in males than in females in MC38 tumors, but the difference was not statistically significant (FIG. 2D). Thus, IHC and flow cytometry confirmed that PD-L1 expression in colon tumors was influenced by sex.

### Example 3

### Confirmation of PD-L1-Expressing Tumor and Tumor-Associated Endothelial Cell Population Proportions by Sex

Next, we analyzed the frequency of tumor-associated cell populations, including tumor cells, tumor-associated immune cells (TAI), tumor-associated macrophages (TAMs), tumor-associated endothelial cells (TECs), and cancer-associated fibroblasts (CAFs), by gating strategy. Then, we analyzed the frequency of PD-L1-expressing cells in each cell population.

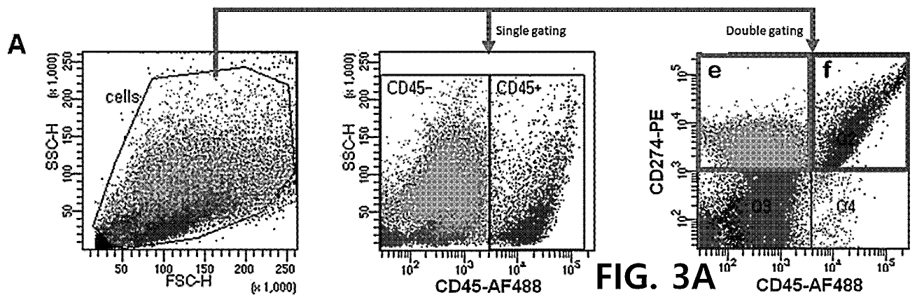

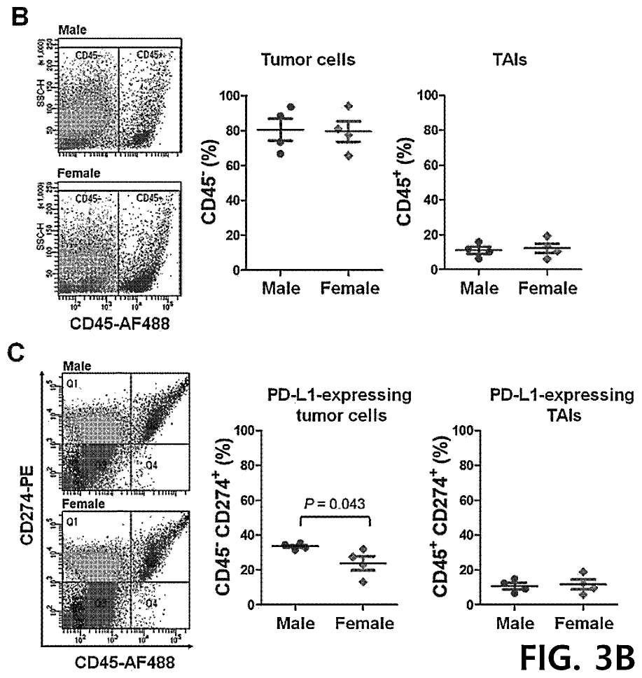

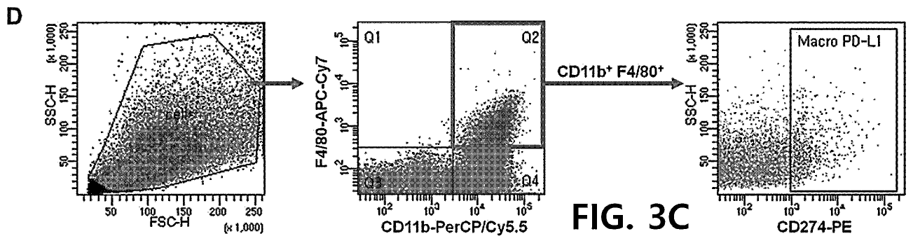

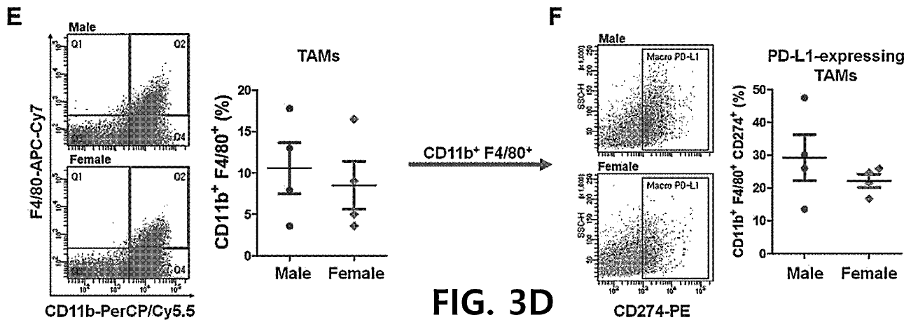

As a result, CD45⁻ and CD45⁺ cells were identified as tumor cells and TAI, respectively (FIG. 3A). Although there was no gender difference in the proportion of CD45⁻ tumor cell population (FIG. 3B, middle panel), PD-L1-expressing tumor cells (CD45⁻CD274⁺) were significantly higher in MC38 tumors from males than from females (FIG. 3C, middle panel, P=0.043). In contrast, there was no gender difference in the proportion of TAI (CD45⁺) (FIG. 3B, right panel) and PD-L1-expressing TAI population (CD45⁺CD274⁺) (FIG. 3C, right panel). The gating strategy for stained cells is shown in FIG. 3C. The proportions of TAMs (CD45⁺CD11b⁺F4/80⁺) and PD-L1-expressing TAMs (CD11b⁺F4/80⁺CD274⁺) did not differ by gender (FIGS. 3E and 3F).

Finally, in the CD45⁻ cell population, CD140a⁻CD31⁺ cells and CD140a⁻CD31⁻ cells were identified as TECs and CAFs, respectively (FIG. 4A). Although there was no gender difference in the proportion of CD140a⁻CD31⁺ TECs in tumors (FIG. 4B, middle panel), PD-L1-expressing TECs (CD140a⁻CD31⁺CD274⁺) were significantly higher in MC38 tumors from males than from females (FIG. 4C, P=0.021). Moreover, the CAF population was significantly higher in MC38 tumors from males than from females (FIG. 4B, right panel; P=0.021), but there were no gender difference with respect to other cell types, such as tumor cells, TAI, TAM, and TEC. However, the proportion of PD-L1-expressing CAF population (CD140a⁻CD31 CD274⁺) did not significantly differ between MC38 tumors from males and females (FIG. 4D).

These results confirmed that a higher proportion of PD-L1-expressing tumors and TECs in MC38 tumors contributed to the aggressive tumor growth in male mice.

### Example 4

### Inhibitory Effect of E2 on MC38 Tumor Growth in Male Mice

Because tumor growth of MC38 cells transplanted into female mice was significantly smaller than that of male mice, we evaluated whether the difference in MC38 tumor growth was due to female sex hormones.

To determine the effect of E2 treatment time, male mice were treated with E2 according to the experimental plan using the MC38 tumor model. A total of 5×10⁵ MC38 cells (passage number 7) resuspended in 100 μl DPBS were injected subcutaneously into the right flank of 8-week-old C57BL/6 male mice. E2 (10 mg/kg) was injected for 1 week from 3 days before injection of MC38 cells (before MC38 group) or from the day of injection MC38 cells (after MC38 group).

Specifically, five mice from each group were used for MC38 cancer cell transplantation, and one mouse from each group that did not develop a tumor was excluded from the experiment. Therefore, in the experiment described in FIG. 5A, results were obtained from 12 out of 15 mice in total. E2 dissolved in olive oil (#E8876, Sigma-Aldrich, St. Louis, MO, USA) was administered intraperitoneally (i.p.) at a concentration of 10 mg/kg seven times daily for 1 week. To determine the effect of E2 treatment time, E2 was injected 3 days before MC38 cell injection (before MC38 group) or on the day of injection (after MC38 group) (FIG. 5A). Mice were sacrificed 20 days after injection of MC38 cells (FIG. 5A), and the collected tumor samples were used for flow cytometry (FIG. 5B).

Tumor growth in mice was monitored 2 to 3 times per week, and tumor size was measured using digital calipers. Tumor volume was calculated using the formula (minimum diameter) 2×(maximum diameter)×0.5. The weight of each resected tumor on the day of sacrifice was measured and used for subsequent analyses.

As a result, MC38 tumor growth was significantly smaller in E2-treated male mice than in control group male mice 18 days after MC38 injection (FIG. 5C; P=0.021). Tumor weights were also significantly lower in E2-treated male mice (before MC38 injection) than in control group male mice (FIG. 5D; P=0.043). In contrast, E2 treatment after MC38 cell injection had no inhibitory effect on tumor growth. These results confirmed that estrogen treatment before MC38 tumor cell transplantation was important for tumor growth inhibition.

### Example 5

Confirmation of the Inhibitory Effect of E2 on PD-L1 Expression and CAF Population in Tumor Cells from Male MC38 Tumors

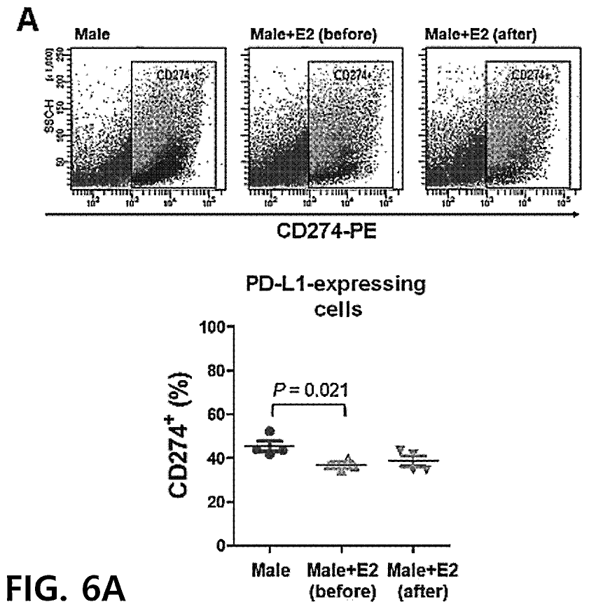

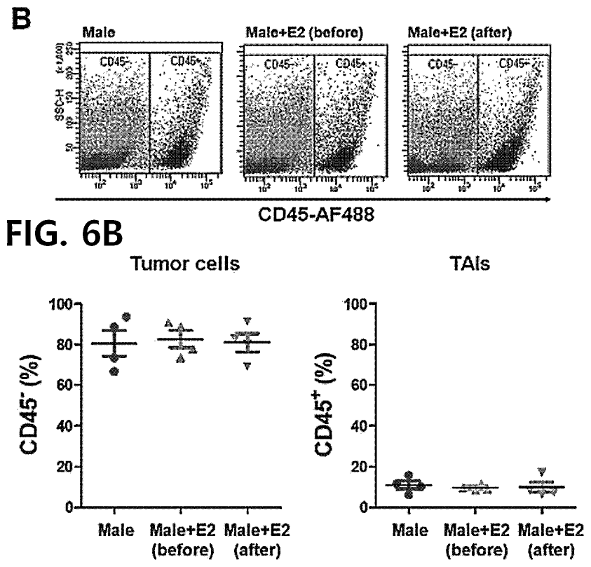

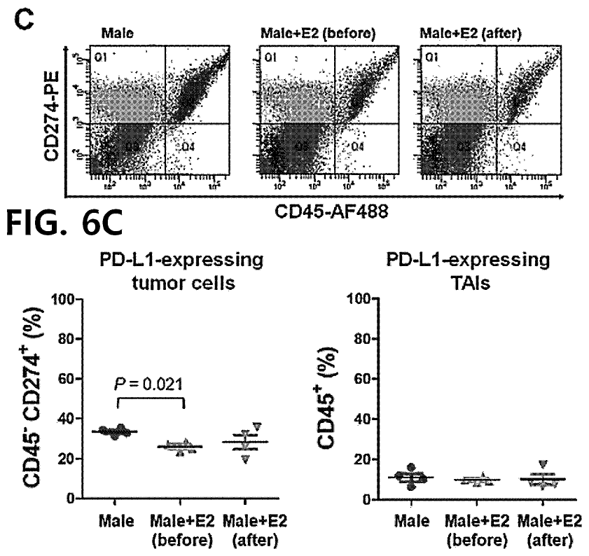

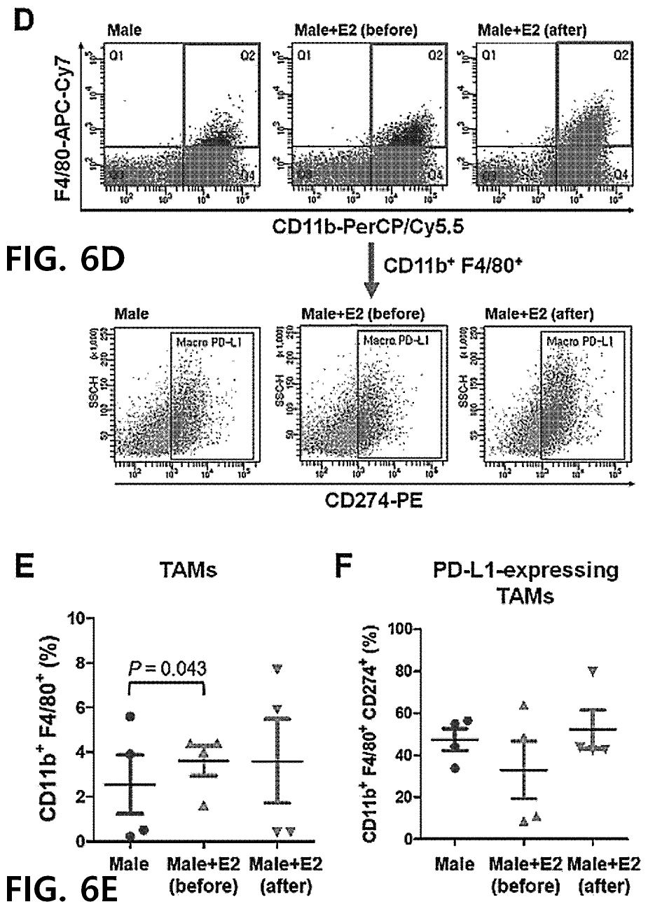

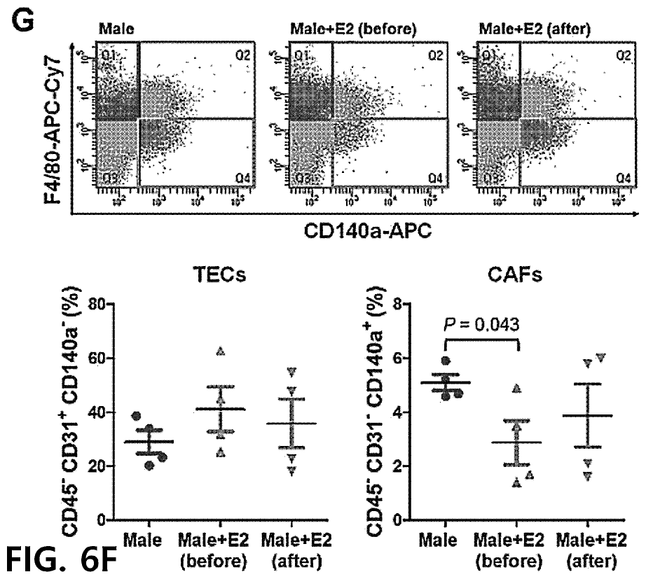

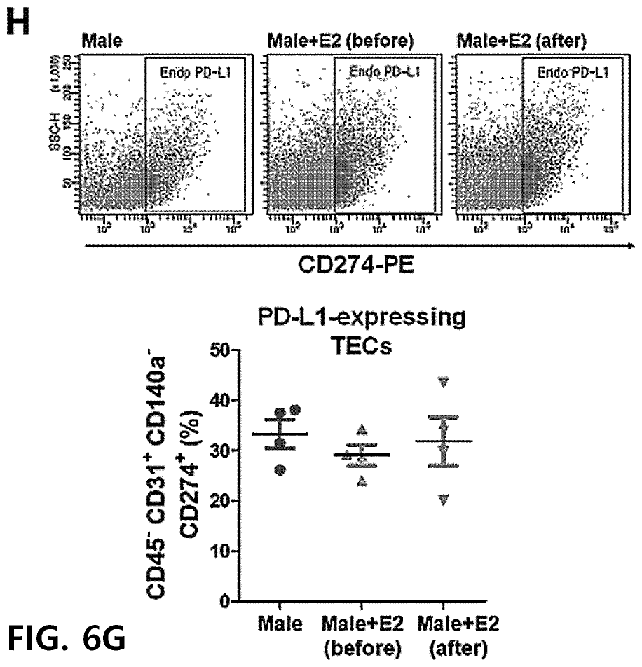

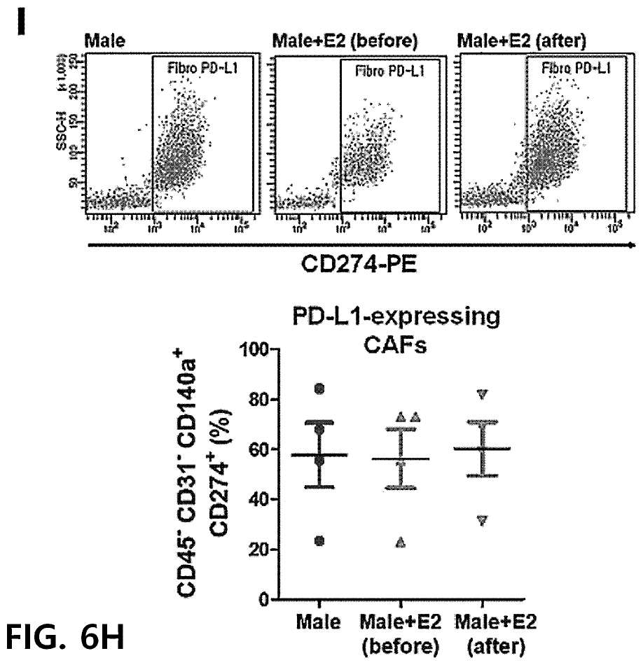

The percentage of PD-L1-expressing cells among total cells was significantly lower in E2-treated male mice (before MC38 injection) than in control groups (FIG. 6A, P=0.021). Moreover, although the percentage of CD45⁻ tumor cell population was not affected by E2 treatment (FIG. 6B, left panel), the expression of PD-L1 on tumor cells was reduced by E2 treatment before MC38 injection (FIG. 6C, left panel; P=0.021). There were no E2 treatment-dependent changes in TAI (FIG. 6B, right panel) or PD-L1-expressing TAI (FIG. 6C, right panel) cell populations. Unexpectedly, the proportion of TAM population increased after E2 treatment compared to the control group (FIG. 6D, top panel and FIG. 6E, P=0.043). However, in the TAM population, PD-L1-expressing cells were reduced after E2 treatment compared to the control group (FIG. 6D, bottom panel and FIG. 6F). TEC and PD-L1-expressing TEC populations increased and decreased, respectively, after E2 treatment compared to the control group (FIG. 6G, left panel and 6H). In addition, although the total CAF population was significantly reduced after E2 treatment compared to the control group (FIG. 6G, right panel; P=0.043), the proportion of PD-L1-expressing cells in the CAF population did not change after E2 treatment (FIG. 6I).

Taken together, these data confirmed that E2 treatment prior to MC38 cell injection into male mice results in changes in cell populations similar to those observed in female mice.

### Example 6

Synergistic Effect of E2 on the Antitumor Effect of Anti-PD-L1 Antibody in MC38 Tumors from Male Mice

Next, we investigated whether there were gender differences in the antitumor effects of anti-PD-L1 antibodies and whether pretreatment E2 enhanced the antitumor effects of anti-PD-L1 antibodies. To this end, mice with tumors measuring 50-100 mm³ were selected from all groups and injected with anti-PD-L1 antibodies.

Specifically, in the experiment described in FIG. 7A, 10 mice from each group were used for MC38 cancer cell transplantation. A total of 5×10⁵ MC38 cells (passage number 8) resuspended in 100 μl DPBS were injected subcutaneously into the right flank of 8-week-old C57BL/6 male mice. After MC38 injection, tumors (50-100 mm³) from male and female mice were selected and used for anti-PD-L1 therapy, and mice with tumors out of range were excluded from this procedure. Therefore, in the experiment described in FIG. 7A, results were obtained from 34 out of 60 mice in total. Mice were injected intraperitoneally every 3 days at a dose of 10 mg/kg with In VivoMAb anti-mouse PD-L1 (B7-H1) (#BE0101, Lot number 751220D1B, concentration 6.6 mg/ml, BioXcell, Clone 10F.9G2) or In VivoMab rat IgG2b isotype control group (#BE0090, Lot number 767920D1, concentration 9.45 mg/ml, BioXcell, Clone LTF-2) antibodies for a total of 3 times.

E2 (10 mg/kg) was administered intraperitoneally for 1 week 3 days before MC38 cell injection. Mice were sacrificed 26 days after MC38 cell injection (FIG. 7A), and at the end of the experiment, MC38 tumor samples were collected and FACS and IHC were performed to evaluate PD-L1 expression (FIG. 7B).

Tumor growth in mice was monitored 2 to 3 times per week, and tumor size was measured using digital calipers. Tumor volume was calculated using the formula (minimum diameter) 2×(maximum diameter)×0.5. The weight of each resected tumor on the day of sacrifice was measured and used for subsequent analyses. The same tumor was divided into two parts to prepare samples for flow cytometry and immunohistochemistry, and experiments were performed.

As a result, MC38 tumor growth was significantly reduced after anti-PD-L1 antibody treatment in male mice, but not in female mice (FIGS. 7C, 7D, and 7E). Moreover, tumor growth was significantly slower (FIG. 7D; P=0.007) and smaller (FIG. 7E; P=0.012) with E2+anti-PD-L1 treatment.

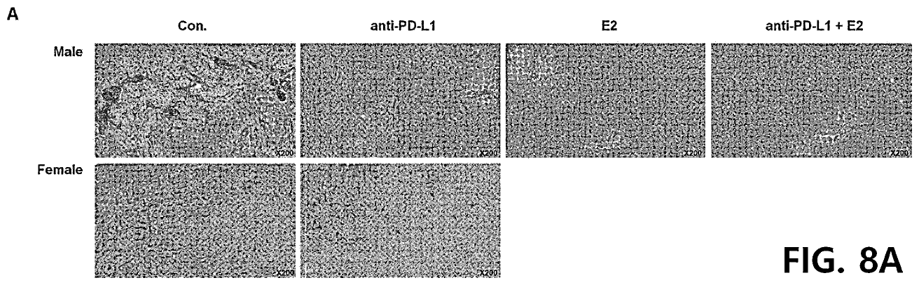

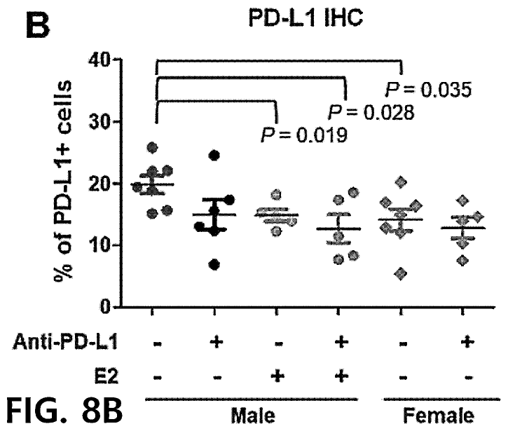

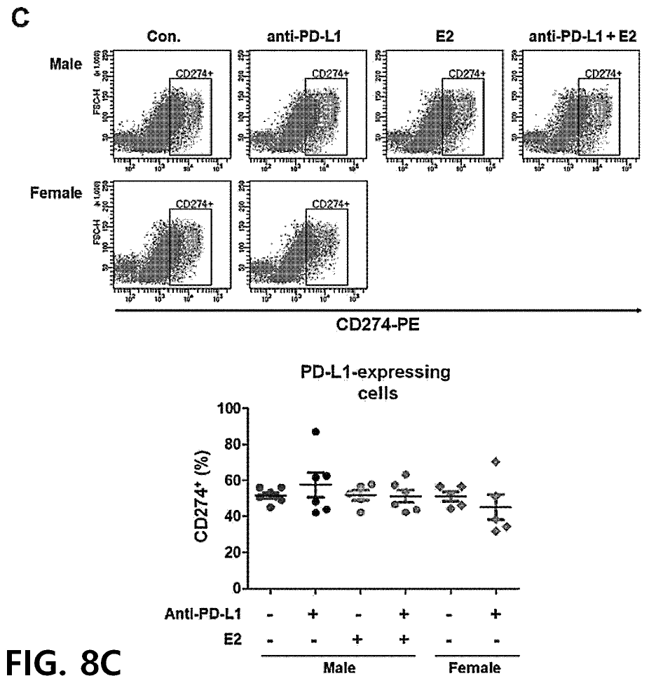

IHC was performed in the same manner as in [Example 2]. IHC results showed that the expression of PD-L1 was significantly lower in MC38 tumor tissues from the male group treated with E2 (P=0.019) or E2+anti-PD-L1 combination (P=0.028) compared with the male control group (FIGS. 8A and 8B). As shown in FIGS. 2A and 2B, PD-L1 expression was significantly lower in female mice than in male mice (FIGS. 8A and 8B; P=0.035). However, the proportion of PD L1-expressing cells did not differ between the groups (FIG. 8C).

Next, PD-L1 expression was analyzed in tumors and immune cells, especially macrophages (FIGS. 9A to 9G). Although there were no differences in the composition of the CD45⁻ tumor cell populations between the groups (FIG. 9A), PD-L1-expressing cells in the tumor cell population were significantly reduced after anti-PD-L1 treatment only in male mice (FIG. 9B, P=0.022). The gating strategy for TAM and M1 TAM is shown in FIG. 9C. Interestingly, in males, co-administration of E2 and anti-PD-L1 resulted in a significant decrease in TAMs (FIG. 9D; P=0.038) and a significant increase in M1 TAMs (CD45⁺CD11b⁺F4/80⁺CD86⁺) (FIG. 9E, P=0.032) compared to E2 or anti-PD-L1 treatment alone. PD-L1-expressing TAMs were significantly reduced after PD-L1 treatment only in females (FIG. 9F; P=0.027). In addition, PD-L1-expressing M1 TAMs were significantly reduced in the E2 alone (P=0.007), anti-PD-L1 alone (P=0.034), and co-therapy groups (P=0.018) compared to the male control group (FIG. 9G).

Taken together, these results confirmed that co-treatment with E2 and anti-PD-L1 potently inhibited MC38 tumor growth and TAMs in male mice compared to treatment with E2 or anti-PD-L1 alone.

1. A pharmaceutical composition for preventing or treating colorectal cancer comprising estrogen and anti-PD-L1 antibody.

2. The pharmaceutical composition for preventing or treating colorectal cancer of claim 1,
wherein the estrogen is any one or more selected from the group consisting of estradiol, estriol, and estrone, and
the anti-PD-L1 antibody is any one or more selected from the group consisting of durvalumab, atezolizumab, avelumab, and MDX-1105.

3. The pharmaceutical composition for preventing or treating colorectal cancer of claim 1,
wherein the pharmaceutical composition is administered by injection, such as subcutaneous injection, intraperitoneal injection, intravenous injection or intramuscular injection.

4. The pharmaceutical composition for preventing or treating colorectal cancer of claim 1,
wherein the weight ratio of the estrogen and the anti-PD-L1 antibody is 0.5 to 1.5:0.5 to 1.5.

5. The pharmaceutical composition for preventing or treating colorectal cancer of claim 1,
wherein the pharmaceutical composition is administered to a male colorectal cancer patient.

6. The pharmaceutical composition for preventing or treating colorectal cancer of claim 1,
wherein the estrogen is administered simultaneously, separately, or sequentially with the anti-PD-L1, or
the estrogen is administered prior to the anti-PD-L1 antibody.

7. The pharmaceutical composition for preventing or treating colorectal cancer of claim 1,
wherein the colorectal cancer is any one or more selected from the group consisting of colon cancer, rectal cancer, colorectal cancer, colon adenocarcinoma, and neuroendocrine tumors.

8. The pharmaceutical composition for preventing or treating colorectal cancer of claim 1,
wherein the estrogen is administered in combination with the anti-PD-L1 antibody to exhibit one or more activities selected from the group consisting of a) to b):
a) reduction of tumor-associated macrophages (TAM); and
b) increase in M1 TAM.

9-16. (canceled)

17. A method for preventing or treating colorectal cancer comprising administering to a subject in need thereof an effective amount of estrogen and an anti-PL-L1 antibody.

18. The method for preventing or treating colorectal cancer of claim 17,
wherein the estrogen is any one or more selected from the group consisting of estradiol, estriol, and estrone, and
the anti-PD-L1 antibody is any one or more selected from the group consisting of durvalumab, atezolizumab, avelumab, and MDX-1105.

19. The method for preventing or treating colorectal cancer of claim 17,
wherein the estrogen and the anti-PL-L1 antibody are administered by injection, such as subcutaneous injection, intraperitoneal injection, intravenous injection or intramuscular injection.

20. The method for preventing or treating colorectal cancer of claim 17,
wherein the weight ratio of the estrogen and the anti-PD-L1 antibody is 0.5 to 1.5:0.5 to 1.5.

21. The method for preventing or treating colorectal cancer of claim 17,
wherein the subject is a male colorectal cancer patient.

22. The method for preventing or treating colorectal cancer of claim 17,
wherein the estrogen is administered simultaneously, separately, or sequentially with the anti-PD-L1 antibody, or
the estrogen is administered prior to the anti-PD-L1 antibody.

23. The method for preventing or treating colorectal cancer of claim 17,
wherein the colorectal cancer is any one or more selected from the group consisting of colon cancer, rectal cancer, colorectal cancer, colon adenocarcinoma, and neuroendocrine tumors.

24. The method for preventing or treating colorectal cancer of claim 17,
wherein the estrogen is administered in combination with the anti-PD-L1 antibody to exhibit one or more activities selected from the group consisting of a) to b):
a) reduction of tumor-associated macrophages (TAM); and
b) increase in M1 TAM.

25-32. (canceled)
